(19) United States
(12) Patent Application Publication
(10) Pub. No.: US 2024/0285964 A1
(43) Pub. Date: Aug. 29, 2024

(54) Systems and Methods for Sensory Entrained Transcranial Magnetic Stimulation

(71) Applicants: The Board of Trustees of the Leland Stanford Junior University, Stanford, CA (US); U.S. Government as represented by the Department of Veterans Affairs, Washington, DC (US)

(72) Inventors: Corey Keller, Redwood City, CA (US); Jessica Ross, Stanford, CA (US)

(73) Assignees: The Board of Trustees of the Leland Stanford Junior University, Stanford, CA (US); U.S. Government as represented by the Department of Veterans Affairs, Washington, DC (US)

(21) Appl. No.: 18/587,541

(22) Filed: Feb. 26, 2024

Related U.S. Application Data
(60) Provisional application No. 63/448,234, filed on Feb. 24, 2023.

(51) Int. Cl. A61N 2/00

(52) U.S. Cl. CPC A61N 2/006

(57) ABSTRACT

Systems and methods for sensory entrained TMS in accordance with embodiments of the invention are illustrated. One embodiment includes a method for transcranial magnetic stimulation, including identifying a beat offset in a music track that when played for a patient is associated with desynchronization of alpha waves and synchronization of beta waves of the patient, playing back the music track for the patient, and providing transcranial magnetic stimulation pulses using a transcranial magnetic stimulation device to the patient, where the pulses are delivered a time equal to the beat offset with respect to beats in the music track.

### CROSS-REFERENCE TO RELATED APPLICATIONS

The current application claims the benefit of and priority under 35 U.S.C. § 119 (e) to U.S. Provisional Patent Application No. 63/448,234 entitled “Systems and Methods for Sensory Entrained Transcranial Magnetic Stimulation” filed Feb. 24, 2023. The disclosure of U.S. Provisional Patent Application No. 63/448,234 is hereby incorporated by reference in its entirety for all purposes.

### FIELD OF THE INVENTION

The present invention generally relates to enhancing the efficacy of transcranial magnetic stimulation using sensory entrainment.

### BACKGROUND

Entrainment in the biomusicological sense refers to the synchronization of organisms to external perceived rhythms, such as those found in music. This synchronization, in humans, extends to brain waves which can change the brain's excitability. In music theory, a beat is the basic unit of time. Rhythm in music is characterized by repeating sequences of stressed and unstressed beats. Groove is a psychological construct used to describe music and its relationship with sensorimotor entrainment. High groove music spontaneously induces a sense of wanting to move, increases spontaneous body movement increases coordinated and distributed muscle activity, improves sensorimotor synchronization to the beat and is consistently perceived and rated by musician and nonmusician listeners, regardless of musical style. Music that induces more sensorimotor coupling (i.e., high in the groove rating) resulted in larger motor-evoked potentials than music with low groove ratings.

Transcranial magnetic stimulation (TMS) is a neuromodulation technique that uses a changing magnetic field to stimulate the brain. There are many different TMS protocols which involve different target structures and different pulse parameters. TMS has been shown to be effective in treating at least depression, obsessive-compulsive disorder, migraines, and addiction.

### SUMMARY OF THE INVENTION

Systems and methods for sensory entrained TMS in accordance with embodiments of the invention are illustrated. One embodiment includes a method for transcranial magnetic stimulation, including identifying a beat offset in a music track that when played for a patient is associated with desynchronization of alpha waves and synchronization of beta waves of the patient, playing back the music track for the patient, and providing transcranial magnetic stimulation pulses using a transcranial magnetic stimulation device to the patient, where the pulses are delivered a time equal to the beat offset with respect to beats in the music track.

In a further embodiment, the music track is selected by playing back each music track of a plurality of music tracks, recording, using electroencephalogram (EEG) electrodes, brain waves of the patient during playback of each music track of the plurality of music tracks, and selecting a music track in the plurality of music tracks that evoked a smallest salience response based on the recorded brain waves of the patient.

In still another embodiment, the plurality of music tracks is constructed using music tracks that match a plurality of musical preferences of the patient.

In a still further embodiment, the plurality of music tracks is further constructed by playing back music tracks that match the plurality of preferences of the patient, recording finger movements of the patient during playback, and selecting the plurality of music tracks based on rhythmic tapping of the patient based on the recorded finger movements.

In yet another embodiment, the music track is played back to the patient using headphones.

In a yet further embodiment, the transcranial magnetic stimulation is single pulse transcranial magnetic stimulation.

In another additional embodiment, the transcranial magnetic stimulation is multi-pulse transcranial magnetic stimulation.

In a further additional embodiment, the beat offset is 200 ms.

In another embodiment again, identifying the beat offset includes playing back the music track, recording, using electroencephalogram (EEG) electrodes, brain waves of the patient during playback of the music track, and identifying a time before a beat onset in the music track at which the brain waves of the patient includes maximum desynchronization of alpha waves and synchronization of beta waves of the patient.

In a further embodiment again, a target of the transcranial magnetic stimulation is in a motor cortex of the patient.

One embodiment includes a system for transcranial magnetic stimulation, including a transcranial magnetic stimulation device, and a controller, including a processor, and a memory, the memory containing a stimulation application that directs the processor to identify a beat offset in a music track that when played for a patient is associated with desynchronization of alpha waves and synchronization of beta waves of the patient, play back the music track for the patient, and provide transcranial magnetic stimulation pulses using the transcranial magnetic stimulation device to the patient, where the pulses are delivered a time equal to the beat offset with respect to beats in the music track.

In still yet another embodiment, to select the music track, the stimulation application further directs the processor to play back each music track of a plurality of music tracks, record, using electroencephalogram (EEG) electrodes, brain waves of the patient during playback of each music track of the plurality of music tracks, and select a music track in the plurality of music tracks that evoked a smallest salience response based on the recorded brain waves of the patient.

In a still yet further embodiment, the stimulation application further directs the processor to construct the plurality of music tracks using music tracks that match a plurality of musical preferences of the patient.

In still another additional embodiment, to construct plurality of music tracks, the stimulation application further directs the processor to play back music tracks that match the plurality of preferences of the patient, record finger movements of the patient during playback, and select the plurality of music tracks based on rhythmic tapping of the patient based on the recorded finger movements.

In a still further additional embodiment, the music track is played back to the patient using headphones.

In still another embodiment again, the transcranial magnetic stimulation is single pulse transcranial magnetic stimulation.

In a still further embodiment again, the transcranial magnetic stimulation is multi-pulse transcranial magnetic stimulation.

In yet another additional embodiment, the beat offset is 200 ms.

In a yet further additional embodiment, to identify the beat offset, the stimulation application directs the processor to play back the music track, record, using electroencephalogram (EEG) electrodes, brain waves of the patient during playback of the music track, and identify a time before a beat onset in the music track at which the brain waves of the patient includes maximum desynchronization of alpha waves and synchronization of beta waves of the patient.

In yet another embodiment again, a target of the transcranial magnetic stimulation is in a motor cortex of the patient. Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1A illustrates a stimulation configuration of a sensory entrained transcranial magnetic stimulation (seTMS) system in accordance with an embodiment of the invention.

FIG. 1B illustrates a personalization configuration of a seTMS system in accordance with an embodiment of the invention.

FIG. 2 is a block diagram for a controller of a seTMS system in accordance with an embodiment of the invention.

FIG. 3 is a process for providing seTMS in accordance with an embodiment of the invention.

FIG. 4 is a process for identifying a pulse timing in accordance with an embodiment of the invention.

FIG. 5 is a series of four charts illustrating enhancement in cortical excitability in two subjects due to seTMS in accordance with an embodiment of the invention.

FIG. 6 is a series of four charts illustrating Mu (alpha band) desynchronization due to seTMS in accordance with an embodiment of the invention.

FIG. 7 is a series of two charts illustrating music induced phase coherence in accordance with an embodiment of the invention.

FIG. 8 conceptually illustrates a single pulse seTMS protocol in accordance with an embodiment of the invention.

FIG. 9 conceptually illustrates a repetitive seTMS protocol in accordance with an embodiment of the invention.

### DETAILED DESCRIPTION

Transcranial magnetic stimulation (TMS) is a method for noninvasive brain stimulation with a rapidly growing use in basic and translational research, as well as clinical medicine. However, a significant amount of heterogeneity in the reported effects of TMS and low test-retest reliability has arisen as more research has been published. In response to this, efforts have been made to optimize TMS methods. One approach is around brain state dependent neuromodulation, where there are optimal brain states to apply TMS and modulate the brain. These brain states can be quantified by the phase of endogenous brain oscillations as measured using electroencephalography (EEG). This approach has shown that when TMS is applied to the primary motor cortex (M1) at times of highest cortical excitability, TMS induces larger brain responses (measured using motor evoked potentials, MEPs). These times of highest cortical excitability can be identified by measuring desynchronizations of a subject's endogenous sensorimotor mu oscillation (μ, ˜10/20 Hz). Sensorimotor μ is associated with inhibitory control, so times of highest cortical excitability correlate with μ being desynchronized and times of lowest cortical excitability correlate with μ being synchronized. These results suggest that applying TMS time-locked with μ waves at times of highest cortical excitability (desynchronized μ) induces larger brain responses than when applied at times of lowest cortical excitability (synchronized μ). This enhanced brain response is reliable and has been reproduced. Moreover, repetitive TMS timed to these brain dynamics enhances changes in excitability (i.e., plasticity) and network changes across connected brain regions.

While these results are promising, phase-aligned TMS currently requires applying TMS pulses according to EEG recordings in real-time, making this technique difficult to implement in many research and clinical settings. Even implementing EEG in clinic visits would require additional preparation time and resources including specialized staff. Further, the technique requires real-time signal processing with high temporal resolution, accurate EEG phase estimation algorithms, and closed-loop TMS-EEG systems. Low cost and low resource alternative solutions are thus needed to increase accessibility to phase-aligned TMS.

In order to provide phase-aligned TMS at low cost in a clinical setting, systems and methods described herein utilize musical beats to phase-align neural oscillations in a patient in order to enhance the effect of TMS in a process referred to herein as sensory entrained TMS (seTMS). Musical rhythms can reproducibly induce desynchronization prior to the musical beat onsets and synchronization after the beat onsets in multiple frequency bands. In many embodiments, instead of timing TMS using real-time EEG recordings, music is used to align the base of cortical oscillations in preparation for TMS. By providing musical events around the TMS pulse, brain oscillations phase-shift to predictable musical beat events. Therefore, the phase dynamics of excitability can be predicted based on the musical track. Once predicted, TMS pulses can be timed when the phase of inhibitory oscillations are desynchronized in order to induce maximum effect. In numerous embodiments, systems and methods described herein enable the personalized identification of specific musical rhythms that provide superior effect for a given patient, as well as personalized pulse stimulation timings for that patient. seTMS systems are described first below, followed by a discussion of seTMS methods.

seTMS Systems

seTMS systems utilize music to induce a brain state that maximizes the efficacy of TMS. In many embodiments, music is played to a patient via headphones, earbuds, or through a loudspeaker. A controller can then synchronize delivery of TMS pulses with the beats of the music track at a predetermined offset from the moment of the beat.

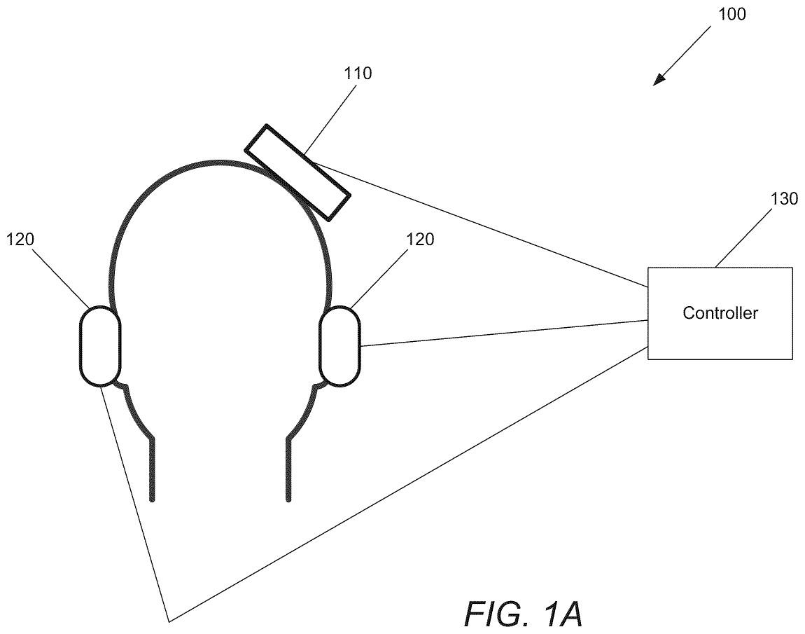

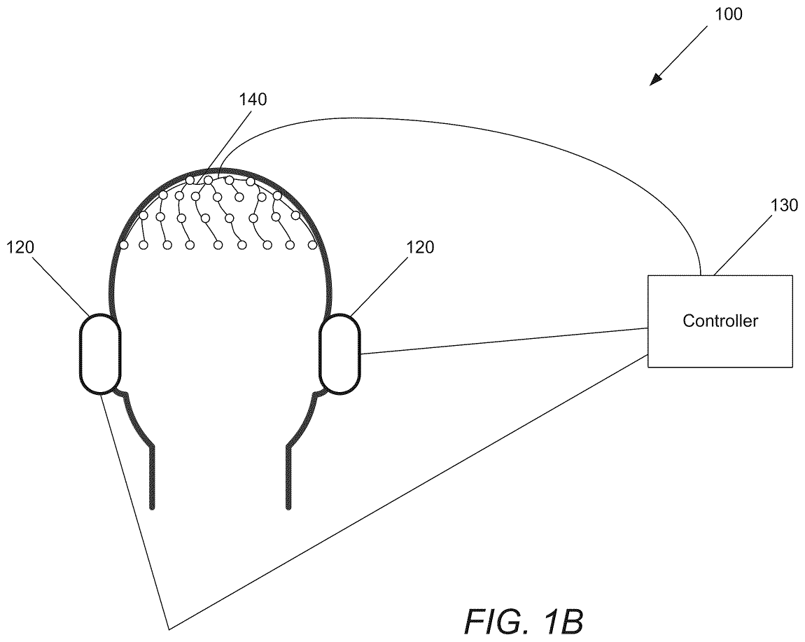

Turning now to FIG. 1A, a seTMS system configured for stimulation in accordance with an embodiment of the invention is illustrated. System 100 includes a TMS device 110. TMS devices are designed to deliver TMS pulses to a predetermined target. Headphones 120 are placed over the user's ears and used to playback the selected music. In numerous embodiments, in-ear headphones (i.e. earbuds) are used. In some embodiments, earmuffs or a different form of noise isolation is provided over the earbuds to reduce distraction from other noise. In various embodiments, loudspeakers are used. A controller 130 synchronizes the TMS device and the music playback such that the TMS pulses are delivered at an offset from the beat of the music playback.

Turning now to FIG. 1B, the seTMS system configured for personalization in accordance with an embodiment of the invention is illustrated. FIG. 1B utilizes EEG electrodes 140 instead of a TMS device. The controller 130 plays back music to the patient via the headphones 120 while recording brain activity using the EEG electrodes 140. In many embodiments, the controller is configured to record the brain activity such that the timing of the music playback is recorded with respect to the timing of the brain activity. The controller can then determine an appropriate offset from the musical beat based on the patient's brain activity to maximize the effectiveness of the TMS.

As can readily be appreciated, FIGS. 1A and 1B represent two specific configurations of a seTMS system. However, different architectures can be used including (but not limited to) those which separate the personalization and stimulation into two separate systems. Further, additional computing hardware can be used to perform many of the computational personalization and/or recording functions instead of the controller without departing from the scope or spirit of the invention. For example, in various embodiments, the controller is merely used as a synchronization circuitry for timing TMS pulses with the music. However, any number of different architectures can be used as appropriate to the requirements of specific applications of embodiments of the invention.

By way of additional example, in numerous embodiments, a personalization configuration may additionally use cameras and/or other methods of motion capture in order to record physical response to music, i.e. finger tapping, foot tapping, etc., which in turn can be used for additional personalization.

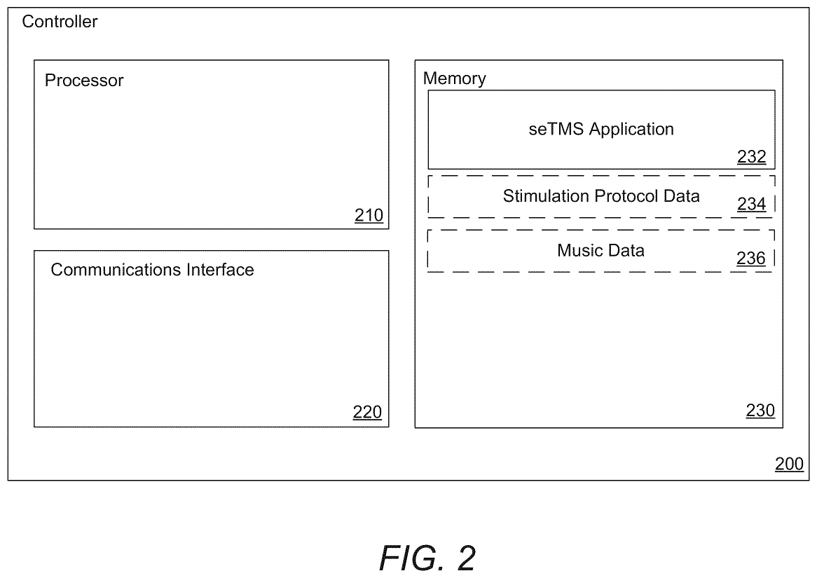

Turning now to FIG. 2, a block diagram for a controller in accordance with an embodiment of the invention is illustrated. Controller 200 includes a processor 210. In many embodiments, the processor is one or more logic processing circuitries including (but not limited to) a central processing unit (CPU), a graphics processing unit (GPU), an application-specific integrated circuit (ASIC), a field-programmable gate array (FPGA), and/or any other logic processing circuitry capable of performing processes described herein as appropriate to the requirements of specific applications of embodiments of the invention. Controller 200 further includes an input/output (I/O) interface 220. In many embodiments, I/O interfaces are used to communicate with other devices such as (but not limited to) TMS devices, headphones, and/or any other connected device as appropriate to the requirements of specific applications of embodiments of the invention.

Controller 200 additionally includes a memory 230. Memories can be made of volatile memory, non-volatile memory, and/or any combination thereof. Memory 230 contains a seTMS application which can configure the processor to carry out various seTMS processes such as (but not limited to) synchronizing TMS pulse delivery with music and/or determining personalized musical tracks and/or beat offsets. Memory 230 at various times may include stimulation protocol data 234 which describes a particular TMS protocol to be delivered, and music data 236 that encodes the music track to be played back.

As can readily be appreciated, different computational platforms can be used to similar effect. Similarly, other sensory input methods besides (or additional to) auditory input can be used with similar effect (i.e. visual, tactile, etc.) to entrain oscillations during TMS. Additionally, while TMS is primarily discussed herein as the neurostimulation modality, other neuromodulatory methods and their associated devices can be used as appropriate to the requirements of specific applications of embodiments of the invention. For example, in various embodiments, sensory entrained versions of focused ultrasound, transcranial direct current stimulation, transcranial alternating current stimulation, deep brain stimulation, vagus nerve stimulation, and/or any other type of neuromodulation can be made to benefit from sensory entrainment methods such as those discussed herein. seTMS methods for stimulation and personalization are described in additional detail below.

seTMS Methods

seTMS stimulation involves delivering TMS pulses in synchrony with a music track, where the TMS pulses are repeatedly offset from the beat of the music. Experimentally, the offset tends to be approximately 200 ms prior to the beat to invoke improved results. However, individuals are idiosyncratic, and therefore the exact timing may change from patient to patient. In order to identify a more optimal timing, a seTMS personalization process can be performed. seTMS stimulation is discussed first followed by personalization.

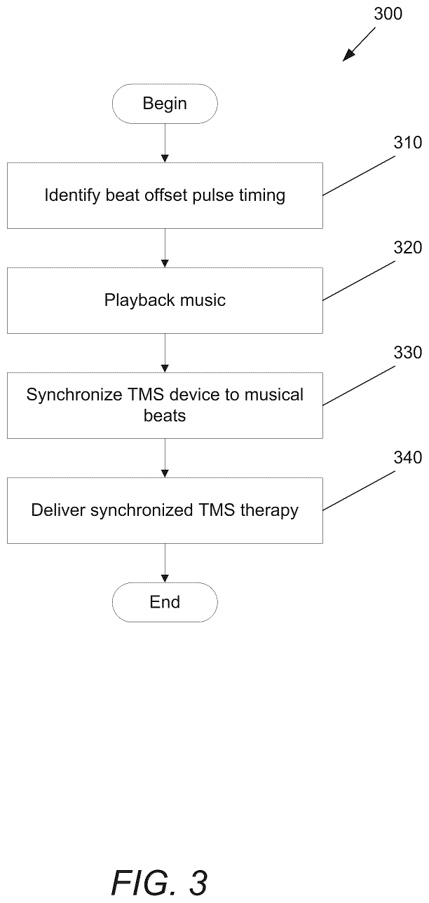

Turning now to FIG. 3, a flow chart for a seTMS stimulation process in accordance with an embodiment of the invention is illustrated. Process 300 includes identifying (310) a beat offset pulse timing. The beat offset timing is the point before the beat at which the patient experiences maximum alpha phase desynchronization and beta frequency phase synchronization. In many embodiments, a default value of 200 ms before the beat is used. In various embodiments, a derived personalized timing is used. As can be readily appreciated, the timing may be greater or less than 200 depending on the given patient. Music is played back (320) to the patient and the TMS device is synchronized (330) to the playback of the music. The TMS therapy is then delivered (340) to the patient as a series of pulses timed with respect to the beat of the music. In many embodiments, pulses may not be delivered offset from every beat. For example, in many embodiments, beats must be at least a predetermined number of seconds apart (e.g. 1-8 s). In this way, pulses will not overlap during high-tempo music.

This method is extensible from single pulse TMS methods to multi-pulse methods. In some embodiments, multi-pulse TMS does not have a fixed pattern. In various embodiments, multi-pulse TMS is theta burst stimulation, and/or any other patterned TMS (e.g. 1 Hz, 10 Hz). In various embodiments, trains may be centered around the beat offset, start at the beat offset, or end at the beat offset.

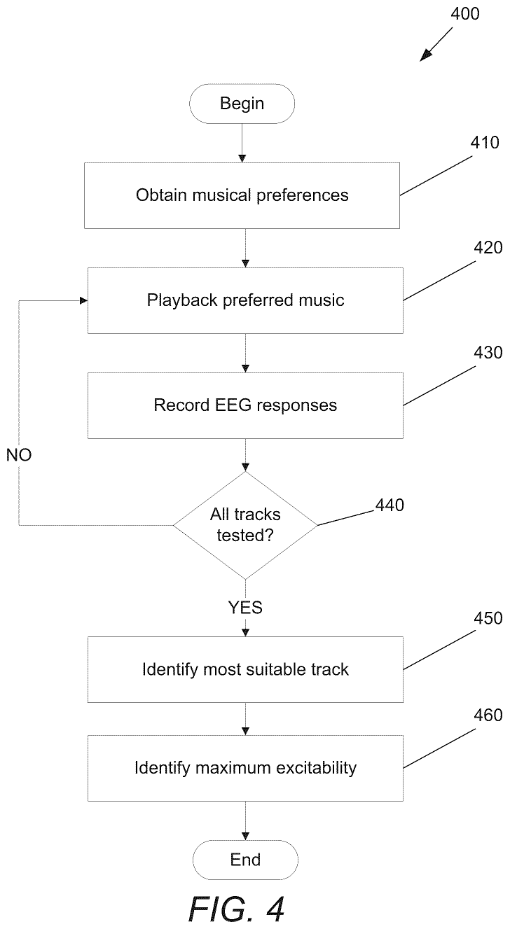

Turning now to FIG. 4, a process for identifying a personalized beat offset in accordance with an embodiment of the invention is illustrated. Process 400 includes obtaining (410) the musical preferences of the patient. In many embodiments, a questionnaire can be used to determine musical genres and/or musical characteristics that the patient prefers in order to select an appropriate set of music for testing. For each given music track, the track is played back (420) to the patient while recording (430) EEG responses.

Once all tracks have been tested (440), the track which is most suitable for seTMS is identified (450). Track suitability is based on a number of factors including (but not limited to) a) enjoyment and familiarity; b) intrinsic timing abilities; and c) beat salience and attention. With respect to perception (a), enjoyment of and familiarity with the musical beats influences brain wave alignment. Self-reported answers from the questionnaire can be used to gather information from subjects for a variety of musical beats. Musical beats are optimal if familiar and enjoyable to the listener. In many embodiments, only tracks that are familiar and enjoyable are tested. However, in some embodiments, tracks that are not explicitly to the patient's preference can be used if no suitable track is found among preferred tracks. With respect to timing ability (b), intrinsic timing abilities also influence brain wave alignment. The patient's ability to tap along with the beats can be recorded. Music that subjects find easy to tap along with will also better align their brain waves, so music is optimal if it minimizes tap timing variability. With respect to salience (c), individual differences in sound processing and attention to musical beats additionally influences brain wave alignment. In many embodiments, the EEG signals recorded during listening to musical beats are analyzed for the strength of sensory responses to musical beats. Sensory responses to salient sounds are present in EEG and can be analyzed for strength of response. Smaller responses indicate that the beat is anticipated, so music is optimal if it induces smaller beat salience responses. In various embodiments, saliency is primarily considered, and perception and timing are not necessarily considered. However, any combination thereof can be considered as appropriate to the requirements of specific applications of embodiments of the invention.

Once a track has been selected, a beat offset is determined by identifying (460) the period of maximum excitability before the beat in the track. In many embodiments, the period of maximum excitability is the maximum state of desynchronization of alpha waves and synchronization of beta waves. In numerous embodiments, the personalization process can be easily performed by playing music for the patient until an appropriate track with an acceptable, repeatable beat offset is determined. Then, during stimulation, the beat offset can be used to direct pulse timings with the understanding that the patient's brain will reproducibly respond.

While seTMS can be personalized as discussed above, performing an EEG is not required. In numerous embodiments, a default beat offset of approximately 200 ms can be used. In this way, seTMS can be performed without having EEG equipment on-site. Similarly, if personalization is performed prior to stimulation, subsequent seTMS can be performed without again performing a personalization process. Additionally, personalization can be performed using other brain wave frequency bands depending on a target stimulation region. For example, if a particular target brain region presents higher excitability when a different brain wave or set of brain waves exhibits a particular pattern, similar methods can be used to entrain that oscillatory behavior.

In numerous embodiments, the EEG signals can be preprocessed. In some embodiments, conservative channel rejection and noise removal is applied to the signals. In various embodiments, missing/removed EEG channels can be interpolated using spherical interpolation, and then the data can be re-referenced to the average. Principal component analysis can be used to reduce the dimensionality of the data, which may further improve decomposition and signal to noise ration of large sources. Artifacts can be removed using any of a variety of methods. For example, fast independent component analysis (fICA) can be performed and the Multiple Artifact Rejection Algorithm (MARA) can then be used to identify components with high likelihood of being non-brain artifacts. While certain preprocessing steps are described above, any number of different signal cleaning methodologies can be applied as appropriate to the requirements of specific applications of embodiments of the invention. Additionally, while certain methods for personalizing music and beat offsets are described above, any number of different modifications can be made such as (but not limited to) performing fewer or additional steps, or performing steps in a different order.

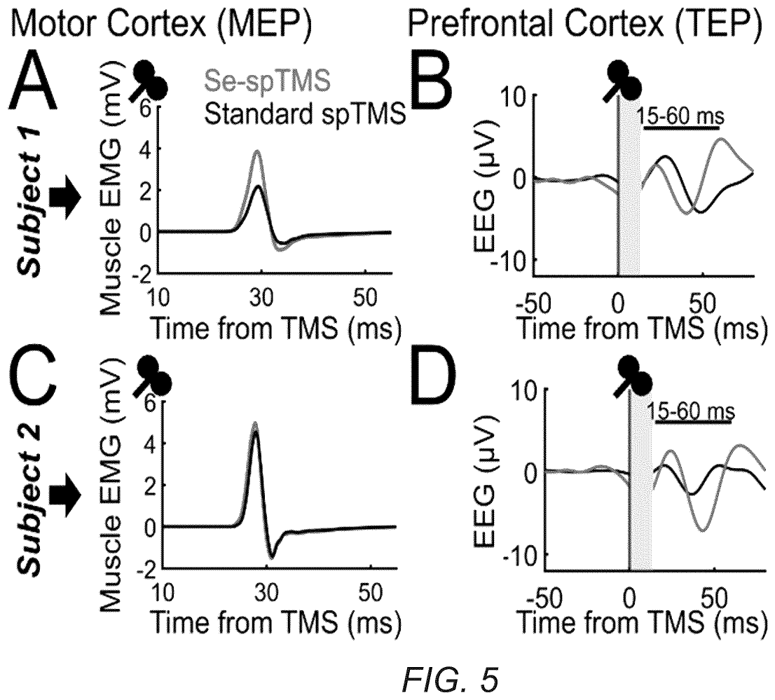

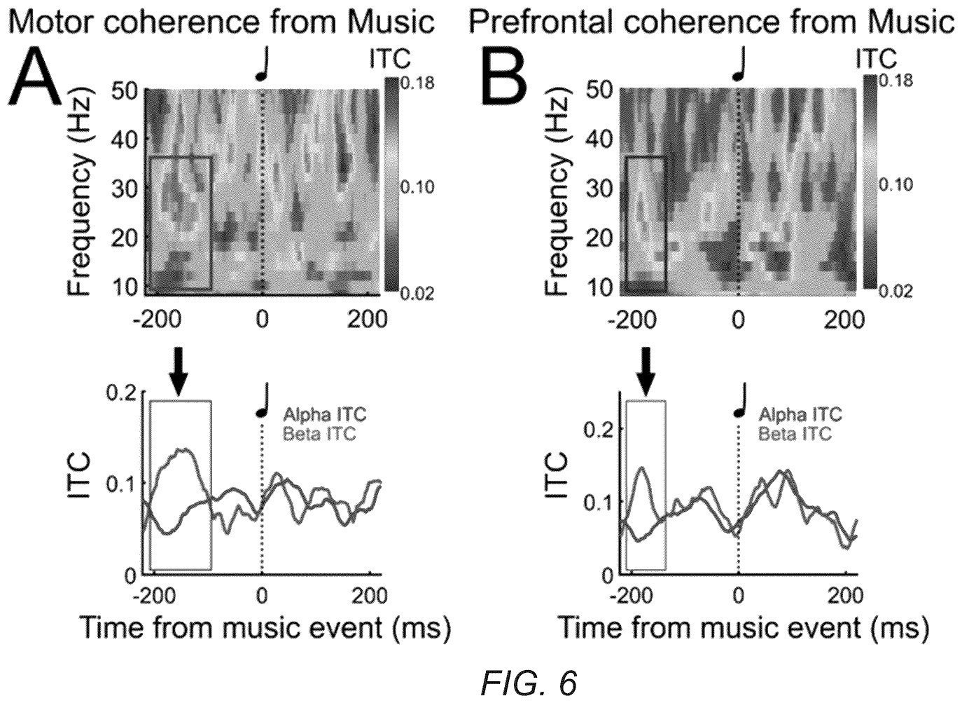

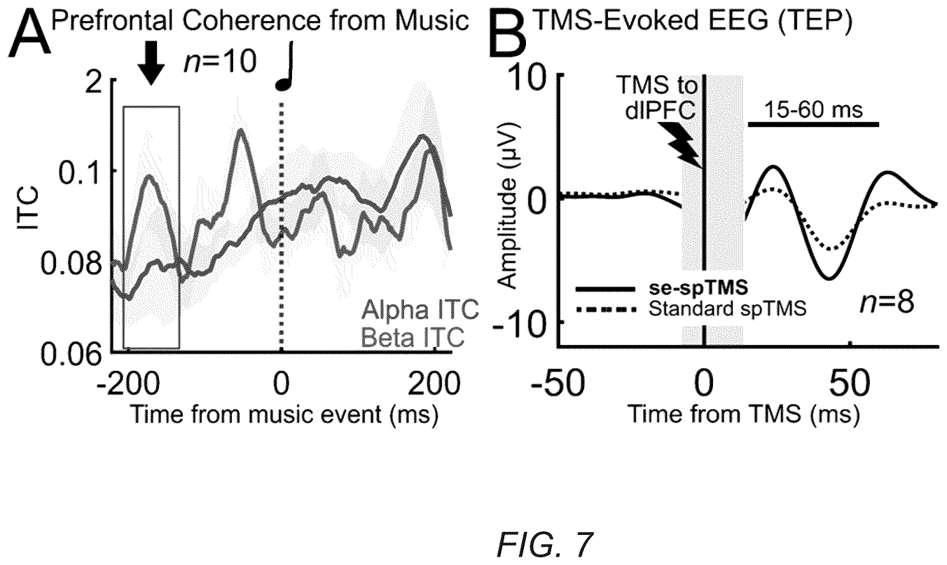

Turning now to FIG. 5, charts illustrating seTMS enhancing cortical excitability in individual subjects in accordance with an embodiment of the invention are illustrated. As can be seen, single pulse seTMS produced increased responses in the primary motor cortex (A,C) based on electromyography data, and in the prefrontal cortex based on EEG data. FIG. 6 illustrates motor cortex and prefrontal cortex coherence induced by music in accordance with an embodiment of the invention by measuring intertrial coherence (ITC). FIG. 7 illustrates prefrontal music-induced phase-coherence across 10 subjects in accordance with an embodiment of the invention (A), and increases in transcranial evoked potential (TEP) increases due to single pulse seTMS compared to traditional single pulse TMS (B).

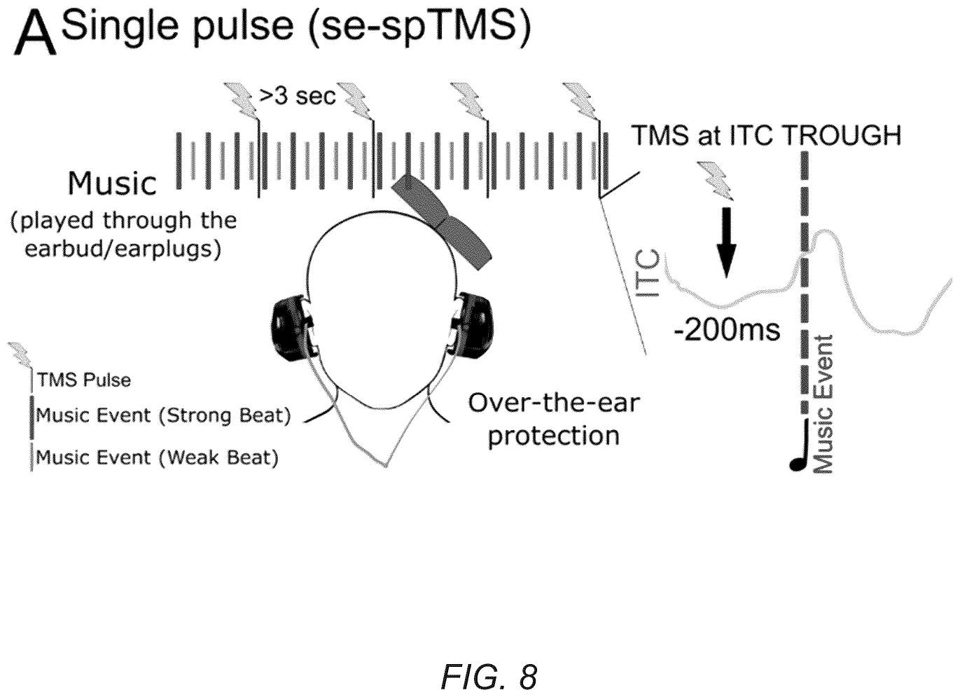

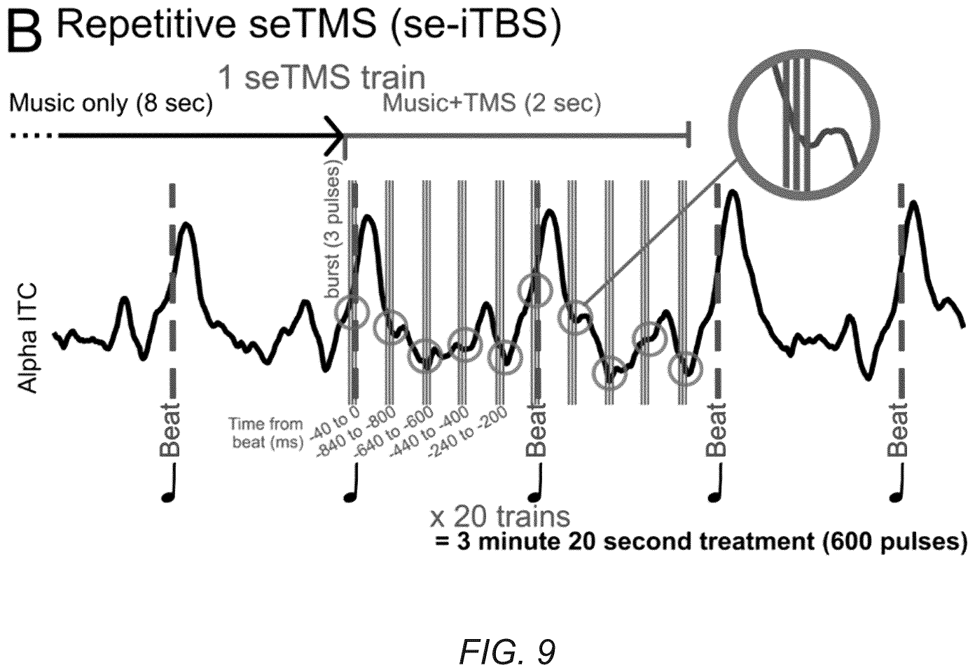

FIG. 8 conceptually illustrates a single pulse seTMS protocol in accordance with an embodiment of the invention. FIG. 9 conceptually illustrates a multi-pulse seTMS protocol in accordance with an embodiment of the invention. As can be readily appreciated, any number of different multi-pulse TMS protocols can be adapted to seTMS protocols by timing the pulse trains to music.

Although specific systems and methods of seTMS are discussed above, many different fabrication methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for transcranial magnetic stimulation, comprising:
identifying a beat offset in a music track that when played for a patient is associated with desynchronization of alpha waves and synchronization of beta waves of the patient;
playing back the music track for the patient; and
providing transcranial magnetic stimulation pulses using a transcranial magnetic stimulation device to the patient, where the pulses are delivered a time equal to the beat offset with respect to beats in the music track.

2. The method of claim 1, wherein the music track is selected by:
playing back each music track of a plurality of music tracks;
recording, using electroencephalogram (EEG) electrodes, brain waves of the patient during playback of each music track of the plurality of music tracks; and
selecting a music track in the plurality of music tracks that evoked a smallest salience response based on the recorded brain waves of the patient.

3. The method of claim 2, where the plurality of music tracks is constructed using music tracks that match a plurality of musical preferences of the patient.

4. The method of claim 3, where the plurality of music tracks is further constructed by:
playing back music tracks that match the plurality of preferences of the patient;
recording finger movements of the patient during playback; and
selecting the plurality of music tracks based on rhythmic tapping of the patient based on the recorded finger movements.

5. The method of claim 1, wherein the music track is played back to the patient using headphones.

6. The method of claim 1, wherein the transcranial magnetic stimulation is single pulse transcranial magnetic stimulation.

7. The method of claim 1, wherein the transcranial magnetic stimulation is multi-pulse transcranial magnetic stimulation.

8. The method of claim 1, wherein the beat offset is 200 ms.

9. The method of claim 1, wherein identifying the beat offset comprises:
playing back the music track;
recording, using electroencephalogram (EEG) electrodes, brain waves of the patient during playback of the music track; and
identifying a time before a beat onset in the music track at which the brain waves of the patient comprise maximum desynchronization of alpha waves and synchronization of beta waves of the patient.

10. The method of claim 1, wherein a target of the transcranial magnetic stimulation is in a motor cortex of the patient.

11. A system for transcranial magnetic stimulation, comprising:
a transcranial magnetic stimulation device; and
a controller, comprising:
a processor; and
a memory, the memory containing a stimulation application that directs the processor to:
identify a beat offset in a music track that when played for a patient is associated with desynchronization of alpha waves and synchronization of beta waves of the patient;
play back the music track for the patient; and
provide transcranial magnetic stimulation pulses using the transcranial magnetic stimulation device to the patient, where the pulses are delivered a time equal to the beat offset with respect to beats in the music track.

12. The system of claim 11, wherein to select the music track, the stimulation application further directs the processor to:
play back each music track of a plurality of music tracks;
record, using electroencephalogram (EEG) electrodes, brain waves of the patient during playback of each music track of the plurality of music tracks; and
select a music track in the plurality of music tracks that evoked a smallest salience response based on the recorded brain waves of the patient.

13. The system of claim 12, where the stimulation application further directs the processor to construct the plurality of music tracks using music tracks that match a plurality of musical preferences of the patient.

14. The system of claim 13, where to construct plurality of music tracks, the stimulation application further directs the processor to:
play back music tracks that match the plurality of preferences of the patient;
record finger movements of the patient during playback; and
select the plurality of music tracks based on rhythmic tapping of the patient based on the recorded finger movements.

15. The system of claim 11, wherein the music track is played back to the patient using headphones.

16. The system of claim 11, wherein the transcranial magnetic stimulation is single pulse transcranial magnetic stimulation.

17. The system of claim 11, wherein the transcranial magnetic stimulation is multi-pulse transcranial magnetic stimulation.

18. The system of claim 11, wherein the beat offset is 200 ms.

19. The method of claim 11, wherein to identify the beat offset, the stimulation application directs the processor to:
play back the music track;
record, using electroencephalogram (EEG) electrodes, brain waves of the patient during playback of the music track; and
identify a time before a beat onset in the music track at which the brain waves of the patient comprise maximum desynchronization of alpha waves and synchronization of beta waves of the patient.

20. The system of claim 11, wherein a target of the transcranial magnetic stimulation is in a motor cortex of the patient.
